(19) United States
(12) Patent Application Publication
(10) Pub. No.: US 2023/0356177 A1
(43) Pub. Date: Nov. 9, 2023

(54) CONVERSION OF CO2 AND H2 TO SYNFUELS

(71) Applicant: HALDOR TOPSØE A/S, Lyngby (DK)

(72) Inventors: Sudip DE SARKAR, KOKKEDAL (DK); Kim AASBERG-PETERSEN, Allerød (DK); Thomas Sandahl CHRISTENSEN, Kgs. Lyngby (DK); Peter Mølgaard MORTENSEN, Roskilde (DK)

(21) Appl. No.: 18/245,730

(22) PCT Filed: Oct. 13, 2021
(86) PCT No.: PCT/EP2021/078304

§ 371 Date: Mar. 17, 2023

(30) Foreign Application Priority Data
Oct. 14, 2020 (EP) 20201822.2
Jul. 15, 2021 (EP) 21185825.3

(51) Int. Cl. B01J 19/24; B01J 19/00; C01B 3/12; C01B 3/34; C07C 1/12; C10L 1/04; C25B 1/042; C25B 1/23; C25B 15/08

(52) U.S. Cl. CPC B01J 19/245; B01J 19/2445; B01J 19/0013; C01B 3/12; C01B 3/34; C07C 1/12; C10L 1/04; C25B 1/042; C25B 1/23; C25B 15/081; B01J 2219/00135; C01B 2203/0283; C01B 2203/0244; C01B 2203/0233; C01B 2203/1235; C01B 2203/062

(57) ABSTRACT

A plant, such as a hydrocarbon plant, is provided, which has a syngas stage (A) for syngas generation and a synthesis stage (B) where said syngas is synthesized to produce syngas derived product, such as hydrocarbon product. The syngas stage (A) primarily includes electrically heated reverse water gas shift (e-RWGS) section. Additionally, an electrically-heated steam methane reforming (e-SMR) section (II) can be arranged in parallel to the e-RWGS section (I). The plant makes effective use of various streams; in particular CO2 and H2. A method for producing a product stream, such as a hydrocarbon product stream is also provided.

### TECHNICAL FIELD

The present invention relates to a plant, such as a hydrocarbon plant, with effective use of various streams, in particular carbon dioxide. A method for producing a product stream, such as a hydrocarbon product stream is also provided. The plant and method of the present invention provide overall better utilization of carbon dioxide.

### BACKGROUND

Carbon capture and utilization (CCU) has gained more relevance in the light of the rise of atmospheric CO₂ since the Industrial Revolution. In one way of utilizing CO₂, CO₂ and H₂ can be converted to synthesis gas (a gas rich in CO and H₂) which can be converted further to valuable products like alcohols (including methanol), fuels (such as gasoline, jet fuel, kerosene and/or diesel produced for example by the Fischer-Tropsch (F-T) process), and/or olefins etc.

Existing technologies focus primarily on stand-alone reverse Water Gas Shift (RWGS) processes to convert CO₂ and H₂ to synthesis gas. The synthesis gas can subsequently be converted to valuable products in the downstream processes as outlined above. The reverse water gas shift reaction proceeds according to the following reaction:

CO₂(g)+H₂(g)↔CO(g)+H₂O(g) (1)

The RWGS reaction (1) is an endothermic process which requires significant energy input for the desired conversion. High temperatures are needed to obtain sufficient conversion of carbon dioxide into carbon monoxide to make the process economically feasible. However, in traditional reactors, heated combustion of for example natural gas or other combustibles, the temperature of the reacted gas may be limited to for example 850-900° C. Alternatively, high conversions of carbon dioxide can also be obtained by using a high H₂/CO₂-ratio. However, this will often result in a synthesis gas with a (much) too high H₂/CO-ratio for the downstream synthesis. Furthermore, an increasing in the use of hydrogen will increase the cost of hydrogen production.

Traditionally, fossil fuel is used to supply necessary heat to the endothermic process, causing increased CO₂ emission and thereby lower effective CO₂ utilization. In reverse water gas shift reactions, it has previously been the goal to limit methanation to take place in parallel to the reverse water gas shift reaction, which typically is challenged when going to temperatures exceeding 500° C. where the reaction kinetics for methanation increases on catalysts traditionally unreactive for this reaction. Such methanation is an undesired side-reaction, which reduces the yield of the process gas and usually it is attempted to avoid or reduce methanation as much as possible. Undesired by-product formation of, for example methane, take place according to one or both of the methanation reactions:

CO(g)+3H₂(g)↔CH₄(g)+H₂O(g) (2)

CO₂(g)+4H₂(g)↔CH₄(g)+2H₂O(g) (3)

The present invention is based on the reactor type, in which it is possible to operate the reverse water gas shift reaction at such high temperature that it is no longer necessary to avoid the methanation reaction, because most of the methane formed will subsequently be converted into hydrogen, CO₂ and CO in the reverse methanation reaction. Furthermore, any methane present in the feed gas can also be converted into synthesis gas according to the reverse methanation reactions. The present invention is further based on the recognition that a prerequisite for this to be possible is that a catalyst capable of catalyzing both reverse water gas shift and methanation is used.

Technologies relying on the RWGS reaction alone have other disadvantages. In some cases, hydrocarbons may be available as co-feed. An example is the availability of hydrocarbons from a downstream synthesis stage (e.g. a propane and butane rich stream from an F-T stage; tail gas comprising different hydrocarbons from an F-T stage; naphtha stream from an F-T stage. Such hydrocarbons cannot be processed in an RWGS reactor without a catalyst with activity for steam reforming (e.g. the reverse of reaction (2) or (3)). If the hydrocarbon streams from the downstream synthesis stage are not used at least in part for additional production of synthesis gas, the overall process may not be feasible from an economic point of view. The same is the case if a hydrocarbon stream, such as natural gas, is available as co-feed to the plant. The CO₂ and H₂ feed streams may also comprise smaller amounts of hydrocarbons.

Another challenge with an rWGS reactor is the conversion of CO₂ into CO, obviating further reaction of the CO into carbon. This carbon can be in the form of both carbon formed on the catalyst or carbon formed on the inner walls of the reactor metal parts. In the latter case the carbon formation may also be in the form of a corrosion type known as metal dusting. The central carbon forming reactions to consider when converting CO₂ into CO are the Boudouard reaction and CO reduction reactions, respectively given as:

CO(g)+H₂(g)⇄H₂O(g)+C(s) (4)

2CO(g)⇄CO₂(g)+C(s) (5)

Both reactions are exothermic and are consequently favored at lower temperatures. Carbon formation and metal dusting would typically take place at low to moderate temperatures up to 400-800° C. depending upon operating conditions, feed gas composition, feed temperature etc. Especially the CO reduction reaction can be a significant challenge when facilitating the reverse water gas shift reaction regime, as it is the intention of a reverse water gas shift reactor to have little to no H₂O in the feed as this gives a reduced potential for conversion according to the reverse water gas shift reaction. This however also means that the potential for carbon formation in the first part of a reverse water gas shift reactor through the CO reduction reaction is high, because the combination of high H₂ partial pressure and low H₂O partial pressure gives a high driving force for this reaction. In this regard it is an advantage to allow the methanation reaction to take place in parallel according reaction (2) and/or (3).

As it can be seen this reaction both reduces the partial pressure of the formed CO and increases the partial pressure of H₂O, both aspects effectively reducing the potential for the CO reduction reaction to take place. Additionally, the risk of carbon formation on the catalyst from the CO reduction reaction is reduced in the case where the methanation reaction also takes place, because catalyst reaction mechanism perspective adsorbed carbon atoms is an intermediate in the methanation reaction scheme (as described by H. S. Bengaard, J. K. Norskov, J. Sehested, B. S. Clausen, L. P. Nielsen, A. M. Molenbroek, J. R. Rostrup-Nielsen, “Steam Reforming and Graphite Formation on Ni Catalysts”, Journal of Catalysis, Volume 209, Issue 2, 2002, Pages 365-384). This means that any formed C atoms on the catalyst surface can be hydrogenated to methane instead of polymerizing to a carbon layer. This gives an advantage in the design of a functional catalyst.

Additionally, the simultaneous occurrence of methanation in the reverse water gas shift reactor results in release of chemical energy to heat the system and, thereby, a temperature increase because methanation is an exothermic reaction. As the CO reduction reaction also is exothermic, the increase in temperature created by the methanation reaction results in a reduction of the potential for the CO reduction reaction and when the temperature has risen to a certain level no potential for the CO reduction reaction will be present at all. This exact level will be dependent on the specific reactant concentration, inlet temperature, and pressure, but will typically be in the range from 600-800° C. above which the CO reduction reaction will not have a potential to take place. The exotherm generated by the methanation reaction will also give the highest temperature rise at the active site of the catalysts surface on where carbon formation reactions usually take place. Therefore, exotherm from methanation reaction has a positive effect on reduction of the carbon formation potential.

In such configuration, e-RWGS reactor allows increasing temperature from relatively low inlet temperature to a high product gas temperature in the reactor. The methanation reaction ((2) and/or (3)) occurs primarily in the first part of the reactor, while produced methane is converted to CO₂, CO and H₂ in the rest of the reactor when temperature exceeds 600-800° C. Thus, this configuration allows reduction of carbon formation potential (by reducing CO content and increasing H₂O content) in the first part of the reactor, and the lower part of the same reactor converts produced methane back to CO at high temperature without any potential for carbon formation.

To address problems with existing technologies, a novel process of syngas preparation and then, synthesis from the said syngas to syngas derived product(s) from primarily CO₂ and H₂ is presented in this document. The proposed layout has at least the following advantages:
1. CO₂ and H₂ can be converted to syngas with a desired H₂:CO ratio, suitably without using any external hydrocarbon feed to the plant. If needed, one or more hydrocarbon co-feed to the plant can be used as well.
2. High conversion in RWGS reaction can be obtained by using electrically heated reactor.
3. Conversion of any hydrocarbon co-feed streams fed to the syngas stage is possible.
4. Better effective utilization of CO₂ as feed can be achieved,
5. There is no risk of carbon formation or metal dusting from carbon monoxide.

In the following the wording “selective RWGS” shall mean that only the reverse water gas shift reaction takes place either on a catalyst or in a reactor while “non-selective RWGS” shall mean that other reactions such as one or more of the methanation reactions (including also reverse methanation) takes place in addition to reverse water gas shift.

### SUMMARY

A plant is provided, said plant comprising:
a. a syngas stage (A), said syngas stage comprising an electrically heated reverse water gas shift (e-RWGS) section (I), and;
b. a synthesis stage (B).

The plant further comprises:
a first feed comprising hydrogen to the e-RWGS section (I); and a second feed comprising carbon dioxide to the e-RWGS section (I); or
a combined feed (8) comprising hydrogen and carbon dioxide to the e-RWGS section (I);
   wherein said e-RWGS section (I) is arranged to convert at least a portion of said first feed and at least a portion of said second feed—or at least a portion of said combined feed—into a first syngas stream, and feed a syngas stream to the synthesis stage (B), and
   wherein said e-RWGS section comprises a structured catalyst comprising a macroscopic structure of electrically conductive material capable of catalysing both a reverse water gas shift reaction and a methanation reaction.

The plant makes effective use of various streams; in particular CO₂ and H₂. A method for producing a product stream, such as a hydrocarbon product stream is also provided, which uses the plant set out above.

Further details of the technology are provided in the enclosed dependent claims, figures and examples.

### LEGENDS TO THE FIGURES

The technology is illustrated by means of the following schematic illustrations, in which:

FIG. 1 shows a first layout of the invention, wherein syngas stage (A) comprises e-RWGS section (I).

FIG. 1a shows a variation of FIG. 1, with recycle of hydrocarbon-containing streams (3a and 3b) from synthesis stage (B) to the syngas stage (A).

FIG. 2 shows another layout of the invention, where the syngas stage (A) comprises a reforming section (II) arranged in parallel to said e-RWGS section (I).

FIG. 2a shows a variation of FIG. 2, in which the reforming section (II) is an autothermal reforming section (IIa).

FIG. 2b shows a variation of FIG. 2, in which the reforming section (II) is a steam methane reforming section (IIb).

FIG. 2c shows a variation of FIG. 2, in which the reforming section (II) is an electrically heated steam methane reforming section (IIc).

FIG. 3 shows a variation of FIG. 2c, in which first feed (1) comprising H₂ comes from electrolysis section (III).

FIG. 4 shows a layout of the invention, including a component recovery stage (C) between syngas stage (A) and synthesis stage (B).

### DETAILED DISCLOSURE

Unless otherwise specified, any given percentages for gas content are % by volume.

Carbon capture and utilization has gained more attention over the years. The proposed layout provides a solution for CO₂ utilization in presence of H₂ to produce syngas and subsequently, conversion of such syngas to valuable products, such as syngas derived liquid fuel, also known as synfuels. For conversion of CO₂ and H₂ feeds to syngas, primarily electrically heated RWGS (e-RWGS) section is used. In the electrically heated RWGS section, either selective or non-selective RWGS may take place. Additionally, electrically heated steam methane reforming (e-SMR) section can be used in parallel to e-RWGS.

In the present technology, carbon dioxide and hydrogen feeds are primarily processed in an e-RWGS section. Additionally, at least one feed comprising hydrocarbons can be processed in an e-SMR section, parallel to e-RWGS section. In one embodiment a feed comprising hydrocarbons may also be processed in the e-RWGS section.

In this context, the term “feed comprising hydrocarbons” is meant to denote a gas with one or more hydrocarbons and possibly other constituents. Thus, typically feed gas comprising hydrocarbons comprises a hydrocarbon gas, such as CH₄ and optionally also higher hydrocarbons often in relatively small amounts, in addition to various amounts of other gasses. Higher hydrocarbons are components with two or more carbon atoms such as ethane and propane. Examples of “hydrocarbon gas” may be natural gas, town gas, naphtha or a mixture of methane and higher hydrocarbons, biogas or LPG. Hydrocarbons may also be components with other atoms than carbon and hydrogen such as oxygenates. The term “feed gas comprising hydrocarbons” is meant to denote a feed gas comprising a hydrocarbon gas with one or more hydrocarbons mixed with steam, hydrogen and possibly other constituents, such as carbon monoxide, carbon dioxide, nitrogen and argon.

The term “synthesis gas” is meant to denote a gas comprising hydrogen, carbon monoxide and also carbon dioxide and small amounts of other gasses, such as argon, nitrogen, methane, etc.

In a first aspect, a plant is provided, said plant comprising:
a. a syngas stage (A), said syngas stage comprising an electrically heated reverse water gas shift (e-RWGS) section (I), and;
b. a synthesis stage (B).

The plant additionally comprises:
a first feed comprising hydrogen to the e-RWGS section; and
a second feed comprising carbon dioxide to the e-RWGS section

As an alternative to separate first feed and second feed, the plant may comprise a combined feed comprising hydrogen and carbon dioxide to the e-RWGS section (I).

The e-RWGS section (I) is arranged to convert at least a portion of said first feed and at least a portion of said second feed—or at least a portion of said combined feed—into a first syngas stream, and feed a syngas stream (e.g. said first syngas stream) to the synthesis stage (B).

In one aspect, the first feed comprising hydrogen to the e-RWGS section and the second feed comprising carbon dioxide to the e-RWGS section are arranged to be mixed to provide a combined feed which is provided to the e-RWGS section.

The first syngas stream suitably has the following composition (by volume):
40-70% H₂ (dry)
10-40% CO (dry)
2-20% CO₂ (dry)

The first syngas stream may additionally contain other components such as methane, steam, and/or nitrogen.

First Feed

A first feed comprising hydrogen is provided to the syngas stage (A). Suitably, the first feed consists essentially of hydrogen. The first feed of hydrogen is suitably “hydrogen rich” meaning that the major portion of this feed is hydrogen; i.e. over 75%, such as over 85%, preferably over 90%, more preferably over 95%, even more preferably over 99% of this feed is hydrogen. One source of the first feed of hydrogen can be one or more electrolyser units. In addition to hydrogen the first feed may for example comprise steam, nitrogen, argon, carbon monoxide, carbon dioxide, and/or hydrocarbons. In some cases a minor content of oxygen may be present in this feed, typically less than 100 ppm. The first feed suitably comprises only low amounts of hydrocarbon, such as for example less than 5% hydrocarbons or less than 3% hydrocarbons or less than 1% hydrocarbons.

Second Feed

A second feed comprising carbon dioxide is provided to the syngas stage (A). Suitably, the second feed consists essentially of CO₂. The second feed of CO₂ is suitably “CO₂ rich” meaning that the major portion of this feed is CO₂; i.e. over 75%, such as over 85%, preferably over 90%, more preferably over 95%, even more preferably over 99% of this feed is CO₂. One source of the second feed of carbon dioxide can be one or more exhaust stream(s) from one or more chemical plant(s). One source of the second feed of carbon dioxide can also be carbon dioxide captured from one or more process stream(s) or atmospheric air. Another source of the second feed could be CO₂ captured or recovered from the flue gas for example from fired heaters, steam reformers, and/or power plants. The second feed may in addition to CO₂ comprise for example steam, oxygen, nitrogen, oxygenates, amines, ammonia, carbon monoxide, and/or hydrocarbons. The second feed suitably comprises only low amounts of hydrocarbon, such as for example less than 5% hydrocarbons or less than 3% hydrocarbons or less than 1% hydrocarbons.

The second feed comprising carbon dioxide may—alternatively or additionally—be a stream comprising CO and CO₂, which is output from an electrolysis section arranged to convert a feed of CO₂ into a stream comprising CO and CO₂.

In a particular aspect, a portion of a CO₂ stream is fed directly to the syngas stage (A) as said second feed comprising carbon dioxide, while another portion of this CO₂ stream is fed to an electrolysis section, where it is converted to a stream comprising CO and CO₂. The stream comprising CO and CO₂ may then be fed to the syngas stage (A).

Combined Feed

As an alternative to separate first feed and second feed, the plant may comprise a combined feed comprising hydrogen and carbon dioxide to the e-RWGS section (I). Typically, the hydrogen content of this combined feed is between 40 and 80%, preferably between 50 and 70%.

Typically, the carbon dioxide content of this combined feed is between 15 and 50%%, preferably between 20 and 40%. Typically, the carbon monoxide content of this combined feed is between 0 and 10%. Typically, the ratio of hydrogen to carbon dioxide in this combined feed is between 1 and 5, preferably between 2 and 4.

In addition to hydrogen and carbon dioxide, the combined feed may for example comprise steam, nitrogen, argon, carbon monoxide, and/or hydrocarbons. The combined feed suitably comprises only low amounts of hydrocarbon, such as for example less than 5% hydrocarbons or less than 3% hydrocarbons or less than 1% hydrocarbons.

Part of the combined feed may be produced by co-electrolysis of a water/steam feed and a CO₂ feed.

Third Feed

A third feed comprising hydrocarbons, external to the plant may be provided to the syngas stage (A). The third feed may additionally comprise other components such as CO₂ and/or CO and/or H₂ and/or steam and/or other components such as nitrogen and/or argon. Suitably, the third feed consists essentially of hydrocarbons or a mixture of hydrocarbons and steam. The third feed of hydrocarbons is suitably “hydrocarbon rich” meaning that the major portion of this feed is hydrocarbons; i.e. over 25%, e.g. over 50%, e.g. over 75%, such as over 85%, preferably over 90%, more preferably over 95%, even more preferably over 99% of this feed is hydrocarbons. The concentration of hydrocarbons in this third feed is determined prior to steam addition (i.e. determined as “dry concentration”).

An example of such third feed can also be a natural gas stream external to the plant. In one aspect, said third feed comprises one or more hydrocarbons selected from methane, ethane, propane or butanes.

The source of the third stream comprising hydrocarbons is external to the plant. The significance of a stream “external to the plant” is that the origin of the stream is not a recycle stream (or a recycle stream further processed or converted) from any synthesis stage in the plant. Possible sources of a third feed comprising hydrocarbons external to the plant include natural gas, LPG, refinery off-gas, naphtha, and renewables, but other options are also conceivable.

e-RWGS Section

The primary section in the syngas stage (A) is an electrically heated reverse water gas shift (e-RWGS) section. Electrically-heated reverse water gas shift (e-RWGS) uses an electric resistance-heated reactor to perform a more efficient reverse water gas shift process and substantially reduces or preferably avoids the use of fossil fuels as a heat source.

An e-RWGS section is used in the present invention for carrying out the reverse water-gas shift reaction between CO₂ and H₂. In a first embodiment the e-RWGS section suitably comprises:
a structured catalyst comprising a macroscopic structure of electrically conductive material capable of catalysing both reverse water gas shift reaction and methanation reaction, said structured catalyst comprising a macroscopic structure of electrically conductive material, said macroscopic structure supporting a ceramic coating, wherein said ceramic coating supports a catalytically active material (for selective e-RGWS);
a pressure shell housing said structured catalyst; said pressure shell comprising an inlet for letting in said feed and outlet for letting syngas product; wherein said inlet is positioned so that said feed enters said structured catalyst in a first end of said structured catalyst and said syngas product exits said structured catalyst from a second end of said structured catalyst;
a heat insulation layer between said structured catalyst and said pressure shell; and
at least two conductors electrically connected to said structured catalyst and to an electrical power supply placed outside said pressure shell, wherein said electrical power supply is dimensioned to heat at least part of said structured catalyst to a temperature of at least 500° C. by passing an electrical current through said macroscopic structure of electrically conductive material; wherein said at least two conductors are connected to the structured catalyst at a position on the structured catalyst closer to said first end of said structured catalyst than to said second end of said structured catalyst, and wherein the structured catalyst is constructed to direct an electrical current to run from one conductor substantially to the second end of the structured catalyst and return to a second of said at least two conductors, and wherein the structured catalyst has electrically insulating parts arranged to direct the current from one conductor, which is closer to the first end of the structured catalyst than to the second end, towards the second end of the structured catalyst and back to a second conductor closer to the first end of the structured catalyst than to the second end.

In a second embodiment, the e-RWGS section suitably comprises:
a structured catalyst comprising a macroscopic structure of electrically conductive material capable of catalysing both reverse water gas shift reaction and methanation reaction, said structured catalyst comprising a macroscopic structure of electrically conductive material, said macroscopic structure supporting a ceramic coating, wherein said ceramic coating supports a catalytically active material (for non-selective e-RWGS);
optionally a top layer arranged on top of the structured catalyst, comprising pellet catalyst, capable of catalysing both the methanation reaction and the reverse water gas shift reaction (for non-selective e-RWGS);
optionally a bottom layer arranged below the structured catalyst, comprising pellet catalyst, capable of catalysing both the methanation reaction and the reverse water gas shift reaction (for non-selective e-RWGS);
a pressure shell housing said structured catalyst; said pressure shell comprising an inlet for letting in said feed and outlet for letting syngas product; wherein said inlet is positioned so that said feed enters said structured catalyst in a first end of said structured catalyst and said syngas product exits said structured catalyst from a second end of said structured catalyst;
a heat insulation layer between said structured catalyst and said pressure shell; and
at least two conductors electrically connected to said structured catalyst and to an electrical power supply placed outside said pressure shell, wherein said electrical power supply is dimensioned to heat at least part of said structured catalyst to a temperature of at least 500° C. by passing an electrical current through said macroscopic structure of electrically conductive material; wherein said at least two conductors are connected to the structured catalyst at a position on the structured catalyst closer to said first end of said structured catalyst than to said second end of said structured catalyst, and wherein the structured catalyst is constructed to direct an electrical current to run from one conductor substantially to the second end of the structured catalyst and return to a second of said at least two conductors, and wherein the structured catalyst has electrically insulating parts arranged to direct the current from one conductor, which is closer to the first end of the structured catalyst than to the second end, towards the second end of the structured catalyst and back to a second conductor closer to the first end of the structured catalyst than to the second end.

The pressure shell suitably has a design pressure of between 2 and 30 bar. The pressure shell may also have a design pressure of between 30 and 200 bar. The at least two conductors are typically led through the pressure shell in a fitting so that the at least two conductors are electrically insulated from the pressure shell. The pressure shell further comprises one or more inlets close to or in combination with at least one fitting in order to allow a cooling gas to flow over, around, close to, or inside at least one conductor within said pressure shell. The exit temperature of the e-RWGS section (I) is suitably 900° C. or more, preferably 1000° C. or more, even more preferably 1100° C. or more.

In case of non-selective e-RWGS, methanation according to reactions (2) and/or (3) takes place in addition to the RWGS reaction. This has the advantage that the concentration of carbon monoxide internally in the reactor is lower than if only reverse water gas shift takes place. This is especially important in the low to moderate temperature range up to ca. 600-800° C. In this temperature range a potential for carbon formation or metal dusting exists or is significantly larger with a selective RWGS catalyst than with a non-selective catalyst.

In one embodiment, the methanation reaction(s) also occur at and near the inlet of the reactor. However, at a given temperature (depends on the feed gas composition, pressure, catalyst activity, extent of heat supply and other factors) the reverse of the methanation reaction will be thermodynamically favoured. In other words, in the first part of the RWGS reactor methane will be formed and in the second part downstream of the first part methane will be consumed according to the reverse of reactions (2) and/or (3).

In one embodiment of the eRWGS reactor of the invention, the eRWGS reactor comprises a structured catalyst. The said structured catalyst has a first reaction zone disposed closest to the first end of said structured catalyst, wherein the first reaction zone has an overall exothermic reaction, and a second reaction zone disposed closest to the second end of said structured catalyst, wherein the second reaction zone has an overall endothermic reaction. Preferably, said first reaction zone has an extension of between the first 5% to between the first 60% of the length of the total reaction zone in the reactor, wherein reaction zone is understood as the volume of the reactor system catalyzing the methanation and reverse water gas shift reactions as evaluated along the flow path through the catalytic zone.

The combined activity for both reverse water gas shift and methanation in an eRWGS reactor of the invention entails that the reaction scheme inside the reactor will start out as exothermic in the first part of the reactor system but end as endothermic towards the exit of the reactor system. This relates to the heat of reaction (Qᵣ) added or removed during the reaction, according to the general heat balance of the plug flow reactor system:

F·Cₚₘ·dT/dV=Q_{add}+Qᵣ=Q_{add}+Σ(−ΔᵣHᵢ)(−rᵢ)

where F is the flow rate of process gas, Cₚₘ is the heat capacity, V the volume of the reaction zone, T the temperature, Q_{add} the energy supply/removal from the surrounding, and Qᵣ the energy supply/removal associated with chemical reactions which are given as the sum of all chemical reactions facilitated within the volume and calculated as the product between the reaction enthalpy and the rate of reaction of a given reaction.

In one embodiment when using a non-selective RWGS reactor the methane concentration by volume in the gas leaving the e-RWGS reactor is lower than 6% such as lower than 4% or preferably less than 3%. High product gas temperature ensures that the final syngas product has low methane concentration, despite the methane concentration has a peak somewhere along the reaction zone. Therefore, this reactor configuration can operated with none, or little, methane in the feed and only little methane in the product gas, but with a peak in methane concentration inside the reaction zone higher than in the feed and/or product gas. It is advantageous in most cases that the concentration of methane in the synthesis gas is as low as possible as methane does not act as a reactant in downstream synthesis such as methanol or Fischer-Tropsch.

In one embodiment the methane concentration in the e-RWGS section is higher than both the concentration of the inlet gas to the e-RWGS section and the concentration of the exit gas from the e-RWGS section.

The e-RWGS section comprises one or more e-RWGS reactors, and in one embodiment, consists of a single e-RWGS reactor. In this embodiment the methane concentration at (at least) one point inside the reactor may be higher than both the methane concentration of the reactor feed gas and the reactor exit gas.

A low concentration of methane can be achieved by a high temperature out of the e-RWGS reactor. A high temperature has the further advantage that a higher conversion of CO₂ into CO. In an embodiment the exit temperature of the gas from the e-RWGS reactor is higher 900° C., such than higher than 1000° C. or even higher than 1050° C. It is an advantage of the proposed reactor that a higher temperature can be achieved than what is typically possible with an externally fired reactor.

Another means to have a low concentration at the exit of the e-RWGS reactor is to have a low to moderate pressure, such as between 5 and 20 bars or between 8 and 12 bars. In this embodiment the gas leaving the e-RWGS section will typically be cooled and water will be (partially) removed by condensation followed by compression to the desired pressure for downstream applications.

In one embodiment the e-RWGS section is followed by a reforming section (II), which suitably includes an autothermal reformer (ATR). The ATR reactor typically comprises a burner, a combustion chamber, and a catalyst bed contained within a refractory lined pressure shell. In an ATR reactor, partial combustion of the hydrocarbon containing feed by sub-stoichiometric amounts of oxygen is followed by steam reforming of the partially combusted hydrocarbon feed stream in a fixed bed of steam reforming catalyst. Steam reforming also takes place to some extent in the combustion chamber due to the high temperature. The steam reforming reaction is accompanied by the water gas shift reaction. Typically, the gas is at or close to equilibrium at the outlet of the reactor with respect to steam reforming and water gas shift reactions. More details of ATR and a full description can be found in the art such as “Studies in Surface Science and Catalysis, Vol. 152,” Synthesis gas production for FT synthesis“; Chapter 4, p. 258-352, 2004”.”.

In this case, the exit gas from the e-RWGS reactor is directed to an autothermal reformer. In this embodiment the exit gas from the e-RWGS reactor reacts with an oxidant to produce the final synthesis gas. The final synthesis gas in this embodiment typically has a temperature above 950° C., such as above 1020° C., or 1050° C. or above. In this particular embodiment the exit temperature from the e-RWGS reactor will typically be between 600-900° C. such as between 700-850° C. The e-RWGS reactor may in this embodiment either be selective or preferably be non-selective. In one embodiment a feed gas comprising hydrocarbons is added to the exit gas from the e-RWGS reactor upstream of the autothermal reformer. This could for example be tail gas from a downstream Fischer-Tropsch synthesis unit.

In embodiments with an ATR after a non-selective RWGS reactor, the methane concentration leaving the RWGS reactor will preferably be lean, such as less than 20% or preferably less than 12%. A relatively low concentration has the advantage that less oxidant is needed in the autothermal reformer.

In embodiments with an ATR after an RWGS reactor, the gas leaving the RWGS reactor is preferably not cooled (except for heat loss and by mixing with other streams). Cooling of the gas increases the oxygen consumption in the ATR.

The advantage of the embodiment with the ATR is that the power needed for the e-RWGS reactor is reduced due to the lower exit temperature. In one embodiment part or all of the oxygen generated by electrolysis of steam to produce hydrogen for the e-RWGS reactor is used in the autothermal reformer.

The oxidant for the autothermal reformer may either be oxygen, air, a mixture of air and oxygen, or be an oxidant comprising more than 80% oxygen such as more than 90% oxygen. The oxidant may also comprise other components such as steam, nitrogen, and/or Argon. Typically the oxidant in this case will comprise 5-20% steam.

In one embodiment, a reactor may be present upstream the e-RWGS section. This reactor may be adiabatic or cooled and the catalyst will typically be pellet based. Part or all of the first feed and part or all of the second feed are directed to this reactor. In the reactor the RWGS and methanation reactions (1-3) take place. The exit temperature from this reactor is typically in the range between 400-700° C. The effluent from this reactor is fed to the e-RWGS section optionally after cooling and condensation of part of the formed H₂O. This has the advantage that the amount of CO₂ in the effluent from the e-RWGS section will be lower.

In a specific embodiment a gas comprising carbon monoxide, carbon dioxide, hydrogen, and methane is combined with the 3^{rd} feed comprising hydrocarbons (e.g. tail gas or light end hydrocarbons) to the e-RWGS section. Alternatively, the third feed is composed solely of said gas comprising carbon monoxide, carbon dioxide, hydrogen, and methane One example could be a tail gas from a Fischer-Tropsch synthesis section. Such a gas could for example contain:
10-30% CO
20-70% CO₂
10-30% H₂
5-25% CH₄
0.2-10% other hydrocarbons

Such a stream could be added directly to the e-RWGS section. Alternatively, this stream is initially passed through a water gas shift reactor together with steam (reverse of reaction 1 above):

CO+H₂O↔H₂+CO₂ (6)

This reduces the CO-concentration at the inlet to the e-RWGS section reducing the potential for carbon formation.

The effluent from the water gas shift reactor may also be directed to another reactor (higher hydrocarbon removal reactor). This higher hydrocarbon removal reactor may be adiabatic or cooled and the catalyst will typically be pellet based. In this higher hydrocarbon removal reactor, the RWGS reaction (1) (or the shift reaction (6)) and methanation reactions (2-3) or the reverse methanation reactions (depending upon the gas composition, temperature, and pressure) take place. Furthermore, steam reforming of higher hydrocarbons may take place in this reactor:

CₙHₘ+nH₂O→nCO+(m/2+n)H₂ (7)

The conditions of the reactor are preferably adjusted to convert more than 90%, such as more than 95% of the non-methane hydrocarbons present in the feed mixture. Removal or substantial reduction of non-methane hydrocarbons has the advantage that the risk of carbon formation in the e-RWGS reactor(s) in the e-RWGS section is reduced considerably.

The exit temperature from this higher hydrocarbon removal reactor is typically in the range between 400-700° C. The effluent from this reactor is fed to the e-RWGS section optionally after cooling and condensation of part of the formed H₂O. This has the advantage that the amount of CO₂ in the effluent from the e-RWGS section will be lower. The effluent may be mixed with the first feed and the second feed before being fed to the e-RWGS section.

The e-RWGS reactor may further comprise an inner tube in heat exchange relationship with but electrically insulated from the structured catalyst, said inner tube being adapted to withdraw a product gas from the structured catalyst so that the gas flowing through the inner tube is in heat exchange relationship with gas flowing over the structured catalyst. The connection between the structured catalyst and said at least two conductors may be a mechanical connection, a welded connection, a brazed connection or a combination thereof.

The electrically conductive material suitably comprises an 3D printed or extruded and sintered macroscopic structure, said macroscopic structure supporting a ceramic coating, wherein said ceramic coating supports a catalytically active material. The structured catalyst may comprise an array of macroscopic structures electrically connected to each other. The macroscopic structure may have a plurality of parallel channels, a plurality of non-parallel channels and/or a plurality of labyrinthic channels. The reactor typically further comprises a bed of a second catalyst material upstream said structured catalyst within said pressure shell.

In one aspect, the e-RWGS reactor further comprises a catalyst material in the form of catalyst pellets, extrudates or granulates loaded into the channels of said macroscopic structure. The e-RWGS reactor may further comprise a control system arranged to control the electrical power supply to ensure that the temperature of the gas exiting the pressure shell lies in a predetermined range and/or to ensure that the conversion of the feed gas lies in a predetermined range.

As used herein, the term “macroscopic structure” is meant to denote a structure which is large enough to be visible with the naked eye, without magnifying devices. The dimensions of the macroscopic structure are typically in the range of centimeters or even meters. Dimensions of the macroscopic structure are advantageously made to correspond at least partly to the inner dimensions of the pressure shell, saving room for the heat insulation layer and conductors.

A ceramic coating, with or without catalytically active material, may be added directly to a metal surface by wash coating. The wash coating of a metal surface is a well-known process; a description is given in e.g. Cybulski, A., and Moulijn, J. A., Structured catalysts and reactors, Marcel Dekker, Inc, New York, 1998, Chapter 3, and references herein. The ceramic coating may be added to the surface of the macroscopic structure and subsequently the catalytically active material may be added; alternatively, the ceramic coat comprising the catalytically active material is added to the macroscopic structure.

Preferably, the macroscopic structure has been manufactured by extrusion of a mixture of powdered metallic particles and a binder to an extruded structure and subsequent sintering of the extruded structure, thereby providing a material with a high geometric surface area per volume. A ceramic coating, which may contain the catalytically active material, is provided onto the macroscopic structure before a second sintering in an oxidizing atmosphere, in order to form chemical bonds between the ceramic coating and the macroscopic structure. Alternatively, the catalytically active material may be impregnated onto the ceramic coating after the second sintering. When chemical bonds are formed between the ceramic coating and the macroscopic structure, an especially high heat conductivity between the electrically heated macroscopic structure and the catalytically active material supported by the ceramic coating is possible, offering close and nearly direct contact between the heat source and the catalytically active material of the macroscopic structure. Due to close proximity between the heat source and the catalytically active material, the heat transfer is effective, so that the macroscopic structure can be very efficiently heated. A compact reforming reactor in terms of gas processing per reforming reactor volume is thus possible, and therefore the reforming reactor housing the macroscopic structure may be compact. The reforming reactor of the invention does not need a furnace, and this reduces the size of the electrically heated reforming reactor considerably.

Preferably, the conductors are made of different materials than the macroscopic structure. The conductors may for example be of iron, nickel, aluminum, copper, silver, or an alloy thereof. The ceramic coating is an electrically insulating material and will typically have a thickness in the range of around 100 μm, say 10-500 μm. In addition, a catalyst may be placed within the pressure shell and in channels within the macroscopic structure, around the macroscopic structure or upstream and/or downstream the macroscopic structure to support the catalytic function of the macroscopic structure.

In an e-RWGS reactor, the structured catalyst within said reactor system may have a ratio between the area equivalent diameter of a horizontal cross section through the structured catalyst and the height of the structured catalyst in the range from 0.1 to 2.0.

Preferably, the macroscopic structure comprises Fe, Ni, Cu, Co, Cr, Al, Si or an alloy thereof. Such an alloy may comprise further elements, such as Mn, Y, Zr, C, Co, Mo or combinations thereof. Preferably, the catalytically active material is particles having a size from 5 nm to 250 nm. The catalytically active material may e.g. comprise copper, nickel, ruthenium, rhodium, iridium, platinum, cobalt, or a combination thereof. Thus, one possible catalytically active material is a combination of nickel and rhodium and another combination of nickel and iridium. The ceramic coating may for example be an oxide comprising Al, Zr, Mg, Ce and/or Ca. Exemplary coatings are calcium aluminate or a magnesium aluminum spinel. Such a ceramic coating may comprise further elements, such as La, Y, Ti, K, or combinations thereof.

In one aspect of the plant, the ratio of moles of carbon in the third feed comprising hydrocarbons, preferably in the case when the third feed is external to the plant, to the moles of carbon in CO₂ in the second feed is less than 0.3, preferably less than 0.25 and more preferably less than 0.20 or even lower than 0.10.

By use of an e-RWGS section (as compared to a regular, fired RWGS section), it is possible to produce a product gas with low content of CO₂, which is desired for some applications, e.g. F-T synthesis or methanol synthesis, since the high temperature of e-RWGS operation ensures a high conversion of CO₂ to CO.

ATR Section

In one aspect, the syngas stage may comprise an autothermal reforming (ATR) section, comprising one or more autothermal reactors (ATR), and wherein first, second, third, and fourth feeds are fed to said ATR section. As an alternative, at least a portion of the combined feed may be fed to the ATR section. Part or all of the third feed may be desulfurized and prereformed. All feeds are preheated as required. The key part of the ATR section is the ATR reactor. The ATR reactor typically comprises a burner, a combustion chamber, and a catalyst bed contained within a refractory lined pressure shell. In an ATR reactor, partial combustion of the hydrocarbon containing feed by sub-stoichiometric amounts of oxygen is followed by steam reforming of the partially combusted hydrocarbon feed stream in a fixed bed of steam reforming catalyst. Steam reforming also takes place to some extent in the combustion chamber due to the high temperature. The steam reforming reaction is accompanied by the water gas shift reaction. Typically, the gas is at or close to equilibrium at the outlet of the reactor with respect to steam reforming and water gas shift reactions.

Typically, the effluent gas from the ATR reactor has a temperature of 900-1100° C. The effluent gas normally comprises H₂, CO, CO₂, and steam. Other components such as methane, nitrogen, and argon may also be present often in minor amounts. The operating pressure of the ATR reactor will be between 5 and 100 bars or more preferably between 15 and 60 bars.

The syngas stream from the ATR is cooled in a cooling train normally comprising a waste heat boiler(s) (WHB) and one or more additional heat exchangers. The cooling medium in the WHB is (boiler feed) water which is evaporated to steam. The syngas stream is further cooled to below the dew point for example by preheating the utilities and/or partial preheating of one or more feed streams and cooling in air cooler and/or water cooler. Condensed H₂O is taken out as process condensate in a separator to provide a syngas stream with low H₂O content, which is sent to the synthesis stage.

The “ATR section” may be a partial oxidation “PDX” section. A PDX section is similar to an ATR section except for the fact that the ATR reactor is replaced by a PDX reactor. The PDX rector generally comprises a burner and a combustion chamber contained in a refractory lined pressure shell.

The ATR section could also be a catalytic partial oxidation (cPDX) section.

Syngas Stage (A)

The syngas stage of the present invention may advantageously comprise one or more additional sections, other than the e-RWGS section described above.

In one preferred aspect, the syngas stage (A) may comprise a reforming section (II) arranged in parallel to said e-RWGS section (I); wherein said plant comprises a third feed comprising hydrocarbons to said reforming section (II), and wherein said reforming section (II) is arranged to convert at least a portion of said third feed into a second syngas stream.

The second syngas stream may have the following composition (by volume):
40-70% H₂ (dry)
10-30% CO (dry)
2-20% CO₂ (dry)
0.5-5% CH₄

In this aspect, the first syngas stream from the e-RWGS section (I) is arranged to be combined with the second syngas stream from the reforming section (II) to provide a combined syngas stream. This combined syngas stream is arranged to be fed to the synthesis stage (B).

According to this aspect, the reforming section (II) may be selected from the group consisting of an autothermal reforming (ATR) section (IIa), a steam methane reforming (SMR) section (IIb) and an electrically heated steam methane reforming (e-SMR) section (IIc).

In one aspect, the reforming section (II) is an autothermal reforming (ATR) section (IIa). In this aspect, the plant (X) further comprises a fourth feed comprising steam and—optionally—a fifth feed comprising oxygen to the autothermal reforming (ATR) section (IIa). A fourth feed comprising steam will also be required if the reforming section is an SMR or an e-SMR In another aspect, the reforming section is an electrically heated steam methane reforming (e-SMR) section (IIc). In this aspect, the plant (X) does not comprise a feed comprising oxygen to the electrically heated steam methane reforming (e-SMR) section (IIc). With this aspect, overall CO₂ output from the plant can be reduced.

In one aspect, at least a portion of the second feed comprising carbon dioxide is fed to the reforming section (II).

The third feed comprising hydrocarbons may be a natural gas feed.

Synthesis Stage (B)

As noted above, the plant comprises a synthesis stage (B). Suitably, the synthesis stage (B) is arranged to convert said first syngas stream, and optionally said second syngas stream, into at least a product stream and, optionally, a hydrocarbon-containing off-gas stream. It may comprise other process elements, such as—compressor, heat exchanger, separator etc.

Suitably, the syngas stream at the inlet of said synthesis stage (B) has a hydrogen/carbon monoxide ratio in the range 1.00-4.00; preferably 1.50-3.00, more preferably 1.50-2.10.

In particular, the synthesis stage (B) may be a Fischer-Tropsch (F-T) stage arranged to convert said syngas stream into at least a hydrocarbon product stream and a hydrocarbon-containing off-gas stream in the form of an F-T tail gas stream. In this aspect, at least a portion of said hydrocarbon-containing off-gas stream may be fed to the syngas stage (A) as said third feed comprising hydrocarbons or in addition to said third feed comprising hydrocarbons. This increases the overall carbon efficiency.

In another aspect, the synthesis stage (B) comprises a methanol synthesis stage arranged to provide at least a methanol product stream.

Additionally, the ratio of H₂:CO₂ provided at the plant inlet may be between 1.0-9.0, preferably 2.5-8.0, more preferably 3.0-7.0.

A sixth feed of hydrogen may be arranged to be combined with the first syngas stream, upstream the synthesis stage. This allows the required ratio of H₂:CO₂ to be adjusted as required.

In one embodiment, the plant further comprises an electrolysis section (III) arranged to convert water or steam into at least a hydrogen stream and an oxygen stream, and at least a part of said hydrogen stream from the electrolysis section is arranged to be fed to the syngas stage (A) as said first feed. Additionally, at least a part of the hydrogen stream from the electrolysis section can be comprised as the sixth feed of hydrogen. A part or all of the water or steam, fed to electrolysis section (III), may come from syngas stage (A) or synthesis stage (B).

In the instance where the plant comprises a reforming section (II) being an autothermal reforming (ATR) section (IIa), at least a part of the oxygen stream from the electrolysis section is suitably arranged to be fed to the syngas stage (A) as said fifth feed comprising oxygen.

The electrolysis section (III) may also be arranged to convert a feed of CO₂ into a stream comprising CO and CO₂, wherein at least a part of said stream comprising CO and CO₂ from the electrolysis section (III) is arranged to be fed to the syngas stage (A) as at least a portion of said second feed comprising carbon dioxide.

An electrolysis section may also be arranged upstream the eRWGS to convert a feed of CO₂ and a feed of water or steam into part or all of said combined feed comprising hydrogen and carbon dioxide. In other words, a singly electrolysis section converts both a feed of CO₂ and a feed of water/steam into the combined feed.

In an embodiment, the synthesis gas plant further comprises a gas purification unit and/or a prereforming unit upstream the reforming section. The gas purification unit is e.g. a desulfurization unit, such as a hydrodesulfurization unit. This could also be the case if the hydrocarbon feed is provided to the eRWGS section.

In the prereformer, the hydrocarbon gas will, together with steam, and potentially also hydrogen and/or other components such as carbon dioxide, undergo prereforming according to reaction (iv) in a temperature range of ca. 350-550° C. to convert higher hydrocarbons as an initial step in the process, normally taking place downstream the desulfurization step. This removes the risk of carbon formation from higher hydrocarbons on catalyst in the subsequent process steps. Optionally, carbon dioxide or other components may also be mixed with the gas leaving the prereforming step to form the feed gas.

Component Recovery Stage (C)

The composition of the syngas from the syngas stage of the plant can be adjusted in various ways. For instance, the plant may further comprise a carbon dioxide removal section, located between said syngas stage (A) and said synthesis stage (B), and arranged to remove at least part of the carbon dioxide from the syngas stream. In this case, at least a portion of the carbon dioxide removed from the syngas stream in said carbon dioxide removal section may be compressed and fed as part of said second feed (2) to the syngas stage (A). Carbon dioxide removal units can be, but not limited to, an amine-based unit or a membrane unit. Such a layout also improves efficiency.

Furthermore, in another embodiment, the plant may comprise a hydrogen removal section, located between said syngas stage (A) and said synthesis stage (B), arranged to remove at least part of the hydrogen from the syngas stream. In this case, at least a portion of the hydrogen removed from the syngas stream in said hydrogen removal section may be compressed and fed as part of said first feed (1) to the syngas stage (A). Hydrogen removal units can be, but not limited to, pressure swing adsorption (PSA) units or membrane units.

A method for producing a product stream, such as a hydrocarbon stream, is also provided.

The method comprises the steps of:
providing a plant (X) as defined herein;
supplying at least a part of the first feed comprising hydrogen to the e-RWGS section (I); and supplying at least a part of the second feed comprising carbon dioxide to the e-RWGS section (I);
or supplying a combined feed comprising hydrogen and carbon dioxide to the e-RWGS section (I);
optionally, supplying at least a part of the third feed comprising hydrocarbons to the syngas stage (A);
optionally, supplying at least a part of the fourth feed comprising steam to the syngas stage (A);
converting at least a portion of said first feed and at least a portion of said second feed—or at least a portion of said combined feed—into a first syngas stream, in said e-RWGS section (I);
feeding said first syngas stream to the synthesis stage (B);
converting said syngas stream into at least a product stream and—optionally—at least a hydrocarbon-containing off-gas stream in said synthesis stage (B).

In the instance where the syngas stage (A) comprises a reforming section (II) arranged in parallel to e-RWGS section (I), the method suitably comprises the additional steps of
providing a third feed comprising hydrocarbons and a fourth feed comprising steam to said reforming section (II) and converting at least a portion of said third feed into a second syngas stream in said reforming section (II), and
combining said second syngas stream with said first syngas stream to provide a combined syngas stream and
feeding said combined syngas stream to the synthesis stage (B).

In one preferred aspect of the method, at least a portion of the hydrocarbon-containing stream is fed to the reforming section (II) as said third feed comprising hydrocarbons or in addition to said third feed comprising hydrocarbons.

In another aspect of the method the synthesis stage (B) is a Fischer-Tropsch (F-T) stage arranged to convert said syngas stream into at least a hydrocarbon product stream and a hydrocarbon-containing off-gas stream in the form of an F-T tail gas stream.

In one embodiment of the method, the e-RWGS section is followed by a reforming section (II), which suitably includes an autothermal reformer (ATR).

Other aspects and advantages of the method, and embodiments thereof, correspond to the advantages of the plant, and embodiments thereof, and therefore need not be described in further detail here.

### Specific Embodiments

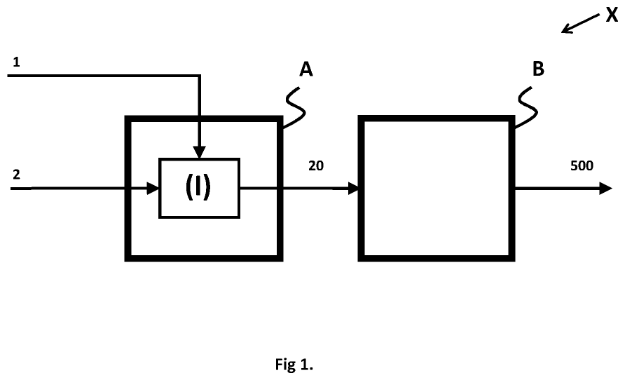

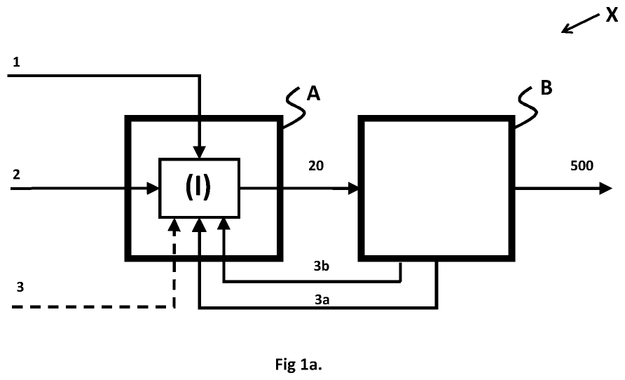

FIG. 1 shows a first layout of the plant of the invention. The plant X comprises a syngas stage (A), and the syngas stage (A) comprises an electrically heated reverse water gas shift (e-RWGS) section (I). The plant also comprises a synthesis stage (B). Plant feeds in FIG. 1 are as follows:
first feed (1) comprising hydrogen to the e-RWGS section (I);
second feed (2) comprising carbon dioxide to the e-RWGS section (I).

The first feed (1) comprising hydrogen, and the second feed (2) comprising carbon dioxide, are supplied to the e-RWGS section (I), which converts them to a first syngas stream (20), and feed said first syngas stream (20) to the synthesis stage (B). The first syngas stream (20) in FIG. 1 is fed to the synthesis stage (B) where it is converted into at least a product stream (500).

FIG. 1a shows a variation of layout in FIG. 1, with recycle of hydrocarbon-containing streams (3a and 3b) from synthesis stage (B) to the syngas stage (A). Stream 3a can be tail gas; 3b can be LPG/naphtha. Optionally, it is possible to take hydrocarbon containing stream (3) from the battery limit.). Also in FIG. 1a, at least a part of the third feed (3) comprising hydrocarbons is supplied to the syngas stage A, in particular to the e-RWGS section (I).

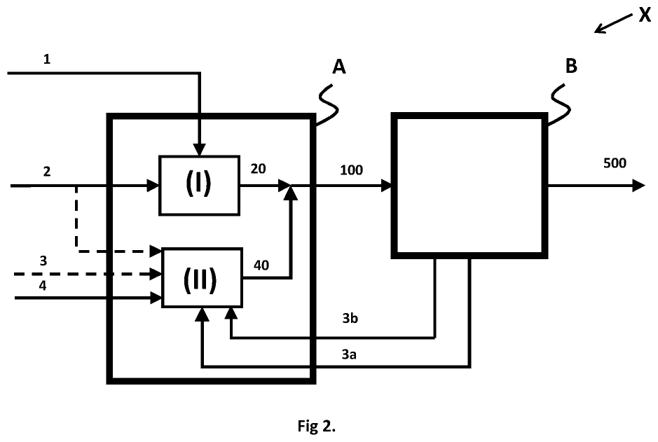

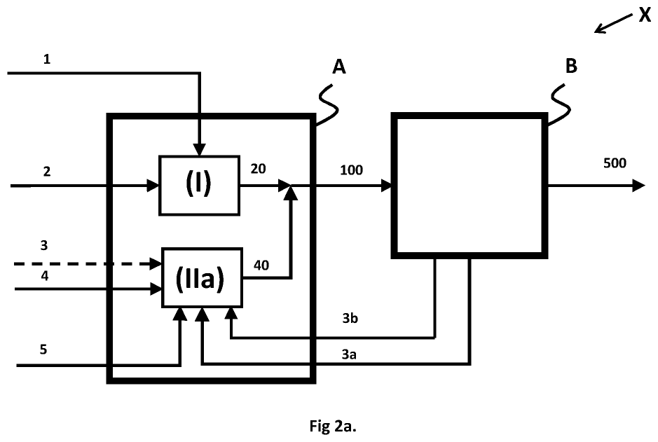

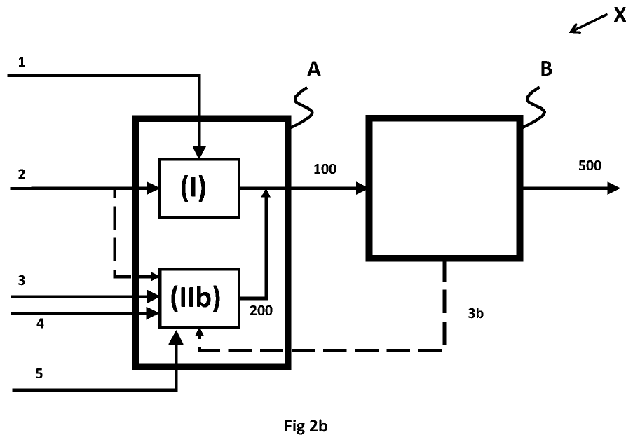

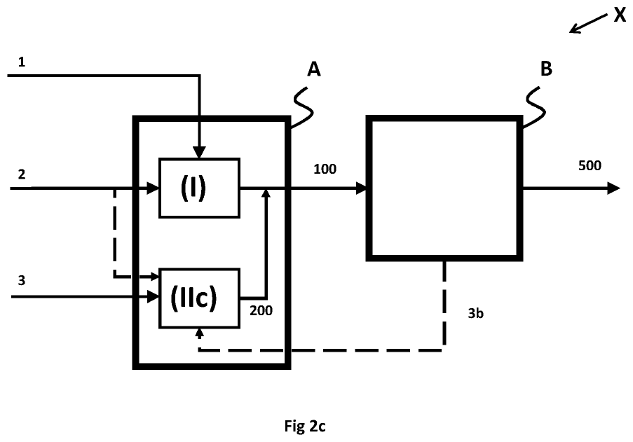

FIG. 2 shows another layout of the invention, where the syngas stage (A) comprises a reforming section (II) arranged in parallel to said e-RWGS section (I). This layout includes an optional third feed (3) comprising hydrocarbons to said reforming section (II). Recycled hydrocarbon streams (3a, 3b) and a fourth feed (4) comprising steam are also fed to the reforming section (II), which is arranged to convert the feeds into a second syngas stream (40). The first syngas stream (20) from the e-RWGS section (I) is combined with the second syngas stream (40) from the reforming section (II) to provide a combined syngas stream (100), and said combined syngas stream (100) is fed to the synthesis stage (B). In the layout illustrated in FIG. 2, a portion of the second feed (2) comprising carbon dioxide may optionally be fed to the reforming section (II).

FIG. 2a shows a variation of the layout in FIG. 2, in which the reforming section (II) is an autothermal reforming section (IIa). In this variation, fifth feed (5) comprising oxygen is fed to the autothermal reforming (ATR) section (IIa).

FIG. 2b shows a variation of the layout in FIG. 2, in which the reforming section (II) is steam methane reforming section (IIb). In this variation, fifth feed (oxygen) is required.

FIG. 2c shows a variation of the layout in FIG. 2, in which the reforming section (II) is an electrically heated steam methane reforming section (IIc).

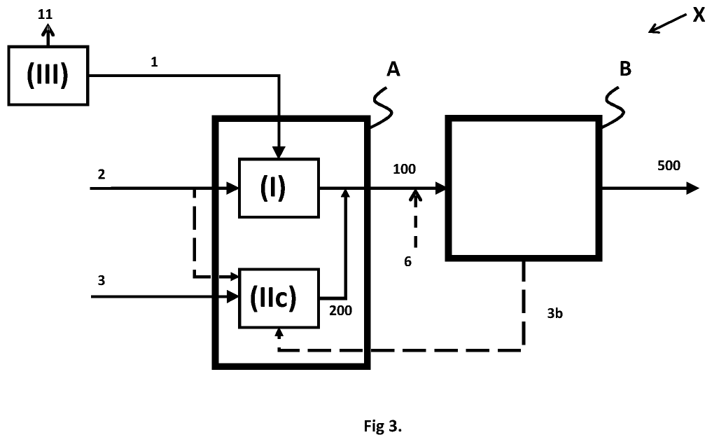

FIG. 3 shows a variation of FIG. 2c, which includes an electrolysis section (III). Electrolysis section (III) converts water or steam (300) into a hydrogen stream and an oxygen stream (11). The hydrogen stream from the electrolysis section is fed to the syngas stage (A) as said first feed (1).

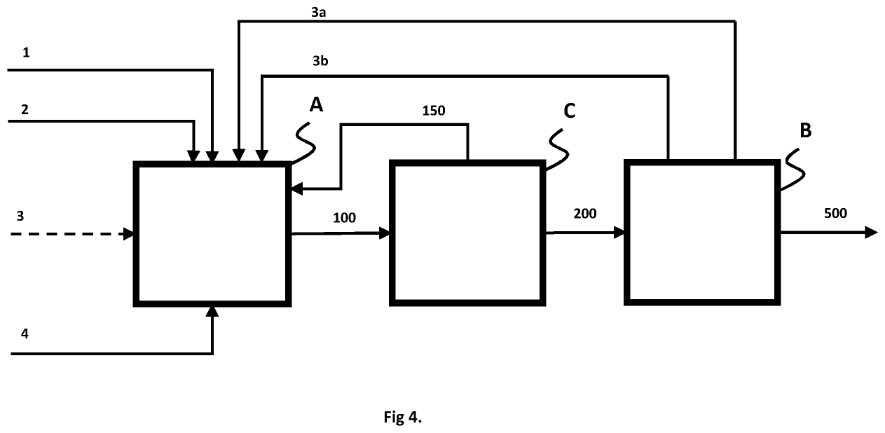

FIG. 4 shows a layout of the invention, including a component recovery stage C, located between syngas stage (A) and synthesis stage (B). Recovered component is recycled (150) to syngas stage (A). Component recovery stage (C) may additionally comprise compressor section (not shown in the figure) where recovered component stream is compressed before recycling.

### LIST OF REFERENCES IN THE FIGURES

A syngas stage
B synthesis stage
C component recovery stage
(I) eRWGS section
(II) reforming section
(IIa) autothermal reforming section
(IIb) steam methane reforming section
(IIc) electrically heated steam methane reforming section
(III) electrolysis section
1 first feed (comprising hydrogen) to syngas stage
2 second feed (carbon dioxide) to syngas stage
3 third feed comprising hydrocarbons, external to the plant
3a first hydrocarbon recycle stream stage A to stage B
3b second hydrocarbon recycle stream stage A to stage B
4 fourth feed comprising steam
5 fifth feed comprising oxygen
6 sixth feed of hydrogen
11 oxygen from electrolysis section (III)
20 first syngas stream
40 second syngas stream
100 combined syngas stream to stage B
150 recycle gas from component recovery stage
200 syngas from component recovery stage
300 water or steam to electrolysis section (III)
500 product from synthesis stage

### EXAMPLES

In this section, the advantages of a novel process for utilization of CO₂-rich feed have been quantified and compared with a conventional plant, based on hydrocarbon feed.

In C1, important process parameters from a conventionally designed syngas stage (A), which consumes primarily hydrocarbon feed, are shown. This syngas stage, comprising an autothermal reformer (ATR) section (Ia), provides syngas to the synthesis stage (B) for production of liquid fuels via Fischer-Tropsch (FT) synthesis. In this example, utilization of CO₂ in a conventional syngas stage has been maximized without compromising the integrity of existing equipment. However, utilization of internal recycle of the hydrocarbon stream from the synthesis stage (B) becomes compromised.

In C2-C4, a H₂ rich feed (1) with CO₂ rich feed (2) have been primarily used as feeds. The layout in syngas stage (A) is based on e-RWGS section (I) in parallel to e-SMR section (IIc). The use of external third feed (3) comprising hydrocarbons is reduced gradually to highlight the flexibility of this layout. Internally recycled hydrocarbon stream comes from synthesis stage (B) which produces liquid fuels based on Fischer-Tropsch synthesis.

TABLE 1

| Parameters | Unit | Ex 1 | Ex 2 | Ex 3 | Ex 4 |
|---|---|---|---|---|---|
| H₂ content in first feed (1) | mol % | 99.0 | 99.0 | 99.0 | 99.0 |
| CO₂ content in second feed (2) | mol % | 99.9 | 99.9 | 99.9 | 99.9 |
| First feed (1)/second feed (2) | — | 2.97 | 3.17 | 2.97 | 2.77 |
| External third feed (3)/second feed (2) | — | 1.34 | 0.64 | 0.13 | 0.00 |
| Fourth feed (4)/first feed (1) | — | 0.31 | 0.64 | 0.28 | 0.15 |
| Fifth feed (5)/first feed (1) | — | 0.38 | — | — | — |
| H₂/CO in syngas product (100) | — | 2.08 | 2.74 | 2.30 | 2.02 |
| Reforming section inlet temperature | ° C. | 650 | 387 | 510 | 583 |
| Reforming section inlet temperature | ° C. | 1025 | 1100 | 1100 | 1100 |
| e-RWGS section inlet temperature | ° C. | — | 250 | 250 | 250 |
| e-RWGS section inlet temperature | ° C. | — | 1100 | 1100 | 1100 |
| CO in syngas product (100)/total C in feeds | % | 74.5 | 81.5 | 80.8 | 80.7 |
| (both external and internal streams) | | | | | |
| Relative CO₂ emission in syngas stage | % | 100 | 0 | 0 | 0 |
| (A)/1000 Nm³ (H₂ + CO) product | | | | | |

In table 1, the relative CO₂ emission is estimated with respect to CO₂ emission in C1 as basis. As it can be seen in C2-C4, no hydrocarbon combustion takes place, causing no CO₂ emission from syngas stage (A). A sustainable source of electricity for the e-RWGS and e-SMR sections are assumed, resulting in no CO₂ emission from these sections.

The examples also demonstrate that the layout is flexible enough to produce syngas with different H₂/CO ratios suitable for downstream synthesis stage (B).

1. A plant (X), said plant comprising:
a. a syngas stage (A), said syngas stage comprising an electrically heated reverse water gas shift (e-RWGS) section (I), and;
b. a synthesis stage (B);
said plant comprising:
a first feed comprising hydrogen to the e-RWGS section (I), and a second feed comprising carbon dioxide to the e-RWGS section (I); or
a combined feed comprising hydrogen and carbon dioxide to the e-RWGS section (I);
wherein said e-RWGS section (I) is arranged to convert at least a portion of said first feed and at least a portion of said second feed—or at least a portion of said combined feed—into a first syngas stream, and feed a syngas stream to the synthesis stage (B), and wherein said e-RWGS section comprises a structured catalyst comprising a macroscopic structure of electrically conductive material capable of catalysing both a reverse water gas shift reaction and a methanation reaction.

2. The plant according to claim 1, wherein said plant (X) additionally comprises a third feed comprising hydrocarbons to the syngas stage (A).

3. The plant according to claim 2, wherein said third feed comprising hydrocarbons is arranged to be fed to the e-RWGS section (I).

4. The plant according to claim 1, wherein said syngas stage (A) comprises a reforming section (II) arranged in parallel to said e-RWGS section (I); wherein said plant comprises a third feed comprising hydrocarbons to said reforming section (II), and wherein said reforming section (II) is arranged to convert at least a portion of said third feed into a second syngas stream, and wherein the first syngas stream from the e-RWGS section (I) is arranged to be combined with said second syngas stream from the reforming section (II) to provide a combined syngas stream, and said combined syngas stream is arranged to be fed to the synthesis stage (B).

5. The plant according to claim 1, wherein said syngas stage (A) comprises a reforming section (II) arranged downstream said e-RWGS section (I); wherein said plant comprises a third feed comprising hydrocarbons to said reforming section (II), and wherein said reforming section (II) is arranged to receive the first syngas stream from the e-RWGS section (I) and provide a second syngas stream, and wherein said second syngas stream is arranged to be fed to the synthesis stage (B).

6. The plant according to claim 1, wherein the content of methane in the synthesis gas stream sent to the synthesis stage (B) is less than 5%.

7. The plant according to claim 2, wherein the ratio of moles of carbon in the third feed comprising hydrocarbons, when external to the plant, to the moles of carbon in CO₂ in the second feed is less than 0.3.

8. The plant according to claim 4, wherein the reforming section (II) is selected from the group consisting of an autothermal reforming (ATR) section (IIa), a steam methane reforming (SMR) section (IIb) and an electrically heated steam methane reforming (e-SMR) section (IIc).

9. The plant according to claim 8, wherein the reforming section (II) is an autothermal reforming (ATR) section (IIa), and wherein the plant (X) further comprises a fourth feed comprising steam and—optionally—a fifth feed comprising oxygen to the autothermal reforming (ATR) section (IIa).

10. The plant according to claim 1, wherein the reforming section is an electrically heated steam methane reforming (e-SMR) section (IIc), and wherein the plant (X) does not comprise a feed comprising oxygen to the electrically heated steam methane reforming (e-SMR) section (IIc).

11. The plant according to claim 4, wherein at least a portion of said second feed comprising carbon dioxide is fed to the reforming section (II).

12. The plant according to claim 1, wherein the operating temperature of the e-RWGS section (I) is 900° C. or more.

13. The plant according to claim 1, wherein the syngas stream at the inlet of said synthesis stage (B) has a hydrogen/carbon monoxide ratio in the range 1.00-4.00.

14. The plant according to claim 1, wherein the ratio of H₂:CO₂ provided at the plant inlet is between 1.0-9.0.

15. The plant according to claim 14, wherein the synthesis stage (B) is an FT synthesis stage and the H₂:CO₂-ratio provided at the plant inlet in the range of 3.0-7.0.

16. The plant according to claim 2, wherein said third feed comprising hydrocarbons is a natural gas feed.

17. The plant according to claim 1, wherein the synthesis stage (B) is arranged to convert said first syngas stream, and optionally said second syngas stream, into at least a product stream and, optionally, internal hydrocarbon-containing streams.

18. The plant according to claim 17, wherein at least a portion of said internal hydrocarbon-containing streams are fed to the syngas stage (A) as said third feed comprising hydrocarbons or in addition to said third feed comprising external hydrocarbons.

19. The plant according to claim 1, wherein the synthesis stage (B) is a Fischer-Tropsch (F-T) stage arranged to convert said syngas stream into at least a hydrocarbon product stream and a hydrocarbon-containing off-gas stream in the form of an F-T tail gas stream.

20. The plant according to claim 1, wherein the synthesis stage (B) is a methanol synthesis stage arranged to convert said syngas stream into at least a hydrocarbon product stream and a hydrocarbon-containing off-gas stream in the form of a methanol product stream.

21. The plant according to claim 1, further comprising an electrolysis section (III) arranged to convert water or steam into at least a hydrogen stream and an oxygen stream, and wherein at least a part of said hydrogen stream from the electrolysis section is arranged to be fed to the syngas stage (A) as at least a portion of said first feed.

22. The plant according to claim 1, comprising a sixth feed of hydrogen arranged to be combined with the syngas stream, upstream the synthesis stage.

23. The plant according to claim 21, wherein at least a part of said hydrogen stream from the electrolysis section is comprised as said sixth feed of hydrogen.

24. The plant according to claim 21, wherein the plant comprises a reforming section (II) being an autothermal reforming (ATR) section (IIa), and wherein at least a part of the oxygen stream from the electrolysis section is arranged to be fed to the syngas stage (A) as said fifth feed comprising oxygen.

25. The plant according to claim 21, wherein the electrolysis section (III) is further arranged to convert a feed of CO₂ into a stream comprising CO and CO₂, and wherein at least a part of said stream comprising CO and CO₂ from the electrolysis section (III) is arranged to be fed to the syngas stage (A) as at least a portion of said second feed (2) comprising carbon dioxide.

26. The plant according to claim 1, wherein the first feed comprising hydrogen and the second feed comprising carbon dioxide are arranged to be mixed to provide a combined feed which is provided to the e-RWGS section.

27. The plant according to claim 1, wherein an electrolysis section is arranged to convert a feed of CO₂ and a feed of water or steam into said combined feed comprising hydrogen and carbon dioxide.

28. A method for producing a product stream, said method comprising the steps of:
providing a plant (X) as defined in claim 1;
supplying at least a part of the first feed comprising hydrogen to the e-RWGS section (I); and supplying at least a part of the second feed comprising carbon dioxide to the e-RWGS section (I);
or supplying a combined feed comprising hydrogen and carbon dioxide to the e-RWGS section (I);
optionally, supplying at least a part of a third feed comprising hydrocarbons, to the e-RWGS section (I);
converting at least a portion of said first feed and at least a portion of said second feed—or at least a portion of said combined feed—into a first syngas stream, in said e-RWGS section (I);
feeding said first syngas stream to the synthesis stage (B);
converting said syngas stream into at least a product stream and—optionally—at least a hydrocarbon-containing off-gas stream in said synthesis stage (B).

29. The method according to claim 28, wherein said syngas stage (A) comprises a reforming section (II) arranged in parallel to e-RWGS section (I), said method comprising the additional steps of:
providing a third feed comprising hydrocarbons to said reforming section (II) and converting at least a portion of said third feed into a second syngas stream in said reforming section (II), and
combining said second syngas stream with said first syngas stream to provide a combined syngas stream and
feeding said combined syngas stream to the synthesis stage (B).

30. The method according to claim 29, wherein, at least a portion of said hydrocarbon-containing off-gas stream is fed to the reforming section (II) as said third feed comprising hydrocarbons or in addition to said third feed comprising hydrocarbons.

31. The method according to claim 28, wherein at least a portion of said third feed comprising hydrocarbon is external to said plant (X).

32. The method according to claim 28 in which the synthesis stage (B) is a Fischer-Tropsch (F-T) stage arranged to convert said syngas stream into at least a hydrocarbon product stream and a hydrocarbon-containing off-gas stream in the form of an F-T tail gas stream.
